(19) United States
(12) Patent Application Publication
(10) Pub. No.: US 2024/0207251 A1
(43) Pub. Date: Jun. 27, 2024

(54) PHARMACEUTICAL COMPOSITION CONTAINING ARTESUNATE OR SALT THEREOF AND PYRONARIDINE OR SALT THEREOF, FOR ANTIPYRESIS, ANTI-INFLAMMATORY EFFICACY, ANTI-VIRAL EFFICACY AND TREATMENT OR PREVENTION OF COVID-19, AND METHOD USING SAME

(71) Applicant: Min Bo SHIM, Seoul (KR)

(72) Inventor: Min Bo SHIM, Seoul (KR)

(21) Appl. No.: 18/557,319

(22) PCT Filed: Apr. 25, 2022
(86) PCT No.: PCT/KR2022/005867

§ 371 Date: Oct. 26, 2023

(30) Foreign Application Priority Data
Apr. 26, 2021 (KR) 10-2021-0053853
Jun. 11, 2021 (KR) 10-2021-0076259

(51) Int. Cl. A61K 31/4745; A61K 31/357; A61P 29/00; A61P 31/14; A61P 31/16

(52) U.S. Cl. CPC A61K 31/4745; A61K 31/357; A61P 29/00; A61P 31/14; A61P 31/16

(57) ABSTRACT

The present invention relates to: a pharmaceutical composition for antipyresis, anti-inflammatory efficacy, anti-viral efficacy and treatment or prevention of COVID-19 infection, the pharmaceutical composition containing artesunate or a salt thereof, and pyronaridine or a salt thereof; and a method using same. Also, the pharmaceutical composition exhibits and excellent antipyretic effect even with a small dosage regimen, has few adverse events, and has an excellent effect in treating and preventing upper respiratory tract infections, COVID-19 and influenza.

### TECHNICAL FIELD

This is on the pharmaceutical composition containing artesunate or salts thereof and pyronaridine or salts thereof for antipyresis, antiinflammation or anti-viral action, or for preventing or treating COVID-19, and method using the same.

### BACKGROUND TECHNOLOGIES

Pyramax® Tab. is a compound formulation containing 60 mg of artesunate and 180 mg of pyronaridine phosphate developed for the treatment of malaria. It has been reported that artesunate and pyronaridine have in vitro anti-viral action on COVID-19 and influenza (Joon-Yong Bae et al., bioRxiv, [Pyronaridine and artesunate are potential antiviral drugs against COVID-19 and influenza], Jul. 28, 2020). Pyramax® Tab. is currently undergoing clinical trial on the treatment of COVID-19 with the same application method/dose as that used for the treatment of malaria. Such application method/dose has been set because it is found necessary to apply the same application method and the dose in excess of that for the treatment of malaria in order to reach the IC₉₀ concentration of pyronaridine and artesunate for SARS-COV 2 in the body as the results of cellular experiment. It is also the same in the case of influenza virus. Application method and dose for the treatment of malaria are as follows:

| | No. of tablets to | Administration |
|---|---|---|
| Body weight | be administered | period |

| | | | |
|---|---|---|---|
| 20~<24 | kg | 1 | 3 days |
| 24~<45 | kg | 2 | |
| 45~<65 | kg | 3 | |
| ≥65 | kg | 4 | |

It was anticipated that the application method and dose used for the treatment of malaria would be optimum for febrile disease or febrile disease accompanied by upper respiratory tract infection.

However, the application method and dose for malaria treatment of Pyramax can cause severe renal and hepatic impairment or hematologic adverse events such as anemia, eosinophilia, neutropenia and increased platelet count, etc.

Therefore, there is a need to develop an administration dose and application method of Pyramax that exhibits optimal preventive or therapeutic effects for febrile disease or febrile disease accompanied by upper respiratory tract infection and has fewer adverse events.

### DETAILED DESCRIPTION OF THE INVENTION

### Technical Tasks

Provide a pharmaceutical composition for antipyresis, anti-inflammation or anti-viral action.

Provide a method for reducing fever using a pharmaceutical composition for antipyresis, anti-inflammation or anti-viral action.

Provide a method of preventing or treating coronavirus infection-19 (COVID-19) or influenza by utilizing a pharmaceutical composition for antipyresis, anti-inflammation or anti-viral action.

### Means of Accomplishing the Task

One aspect is to provide a pharmaceutical composition containing artesunate or a salt thereof and pyronaridine or a salt thereof for antipyresis, anti-inflammation or anti-viral action.

Artesunate can be referred to as dihydroartemisinine-12-alpha-succinate, succinyl dihydroartemisinin and succinyl dihydroartemisinin, (3R,5aS,6R,8aS,9R,10S,12R,12aR)-decahydro-3,6,9-trimethyl-3,12-epoxy-12H-pyrano(4,3-j)-1,2-benzodioxepin-10-ol and hydrogen succinate. The artesunate may be a compound of the CAS No. 88495-63-0 or 88495-63-3. The aforementioned artesunate may be in the form of a stereoisomer, salt, solvate, hydrate, acid, anhydride or free base of artesunate.

Artesunate may be a compound with the following chemical formula:

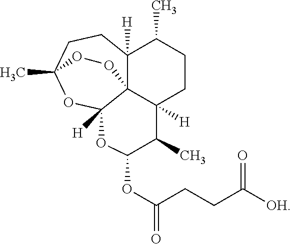 [SMILES: C[C@H]1[C@H](OC(=O)CCC(=O)O)O[C@@H]2O[C@@]3(C)CC[C@H]4[C@H](C)CC[C@@H]1[C@@]24OO3]

Artesunate can be a medicine for treatment of malaria. Artesunate may be administered intravenously, intramuscularly, orally or rectally. Artesunate may induce hemolytic anemia or severe allergic reactions.

pyronaridine can be referred to as 4-[(7-chloro-2-methoxybenzo[b]-1,5-naphthyridin-10-yl)amino]-2,6-bis(1-pyrrolidinylmethyl)phenyl or malaridine. The pyronaridine can be a compound of the CAS number 74847-35-1. The aforementioned pyronaridine can be in the form of a stereoisomer, salt, solvate, hydrate, acid, anhydride or free base of pyronaridine.

The pyronaridine can be a compound of the following chemical formula:

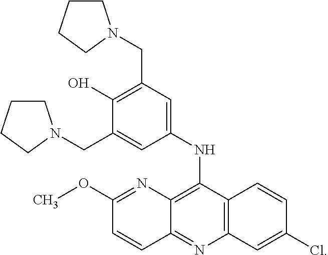 [SMILES: COc1ccc2nc3cc(Cl)ccc3c(Nc3cc(CN4CCCC4)c(O)c(CN4CCCC4)c3)c2n1]

Pyronaridine can be a medicine for the treatment for malaria, cancer or Ebola. Pyronaridine can be administered orally, intramuscularly or intravenously.

The term “salt” refers to an adduct salt of an inorganic, organic or metallic salt of a compound. The aforementioned salt can be a pharmaceutically allowable salt. The aforementioned pharmaceutically allowable salt can be a salt that does not induce significant irritation to the organism for which the compound is administered and does not damage the biological activity and properties of the compound. The aforementioned inorganic salt can be a hydrochloride, bromide, phosphate, sulfate or disulfate. The aforementioned organic salts can be formate, acetate, propionate, lactate, oxalate, tartrate, malate, maleate, citrate, fumarate, besylate, camsylate, edisylate, trichloroacetic acid, trifluoroacetate, benzoate, gluconate, methanesulfonate, glycolate, succinate, 4-toluenesulfonate, galacturonate, embonate, glutamate, methanesulfonic acid, ethanesulfonic acid, benzenesulfonic acid, p-toluenesulfonic acid or aspartate.

The aforementioned metal salt can be calcium salt, sodium salt, magnesium salt, strontium salt or potassium salt.

The aforementioned salt of artesunate can be the sodium salt of artesunate.

The aforementioned salt of pyronaridine can be phosphate, sulfate, hydrochloride, acetate, methanesulfonate, benzenesulfonate, toluenesulfonate, maleate or fumarate of pyronaridine. The aforementioned pyronaridine phosphate can be pyronaridine tetraphosphate (tetraphosphate).

The term “antipyresis” refers to any act that causes an improvement of or beneficial change in the body temperature of an individual to the normal range. Antipyresis refers to the act of lowering the body temperature of an individual with fever or preventing the normal body temperature from dropping. Fever refers to a condition in which the body temperature rises above the normal range. In the case of humans, a person is diagnosed with fever if one or more of the following criteria are met: anorectal or tympanic temperature of more than about 38.0° C., oral temperature of more than about 37.5° C. and armpit temperature of more than about 37.2ºC.

The term “antiinflammation” refers to the ability to suppress or eliminate inflammation. The aforementioned inflammation can be viral inflammation.

The term “anti-viral” refers to the ability to kill or suppress the growth of virus.

The aforementioned pharmaceutical composition can contain effective dose of artesunate or a salt thereof and pyronaridine or a salt thereof. The term “effective dose” refers to a dose sufficient to produce a preventive or therapeutic effect when administered to an individual in need of such prevention or treatment. The aforementioned effective dose can be selected appropriately in accordance with the cell or entity by the corresponding manufacturer. The said effective dose can be determined by factors including the severity of the disease, the age, weight, state of health, gender and sensitivity to the drug of the patient, time and route of administration, discharge ratio, duration of treatment, preparation and formulation used or drugs used concurrently, and other factors that are well known in the medical field. The aforementioned effective doses range from about 1 μg to about 2 g, about 10 μg to about 1 g, about 100 μg to about 1 g, about 1 mg to about 1 g, about 5 mg to about 900 mg, about 10 mg to about 800 mg, about 20 mg to about 700 mg, about 30 mg to about 600 mg, about 30 mg to about 500 mg, about 30 mg to about 400 mg, about 30 mg to about 300 mg, about 30 mg to about 250 mg, about 30 mg to about 240 mg, about 40 mg to about 1 g, about 50 mg to about 900 mg, about 60 mg to about 800 mg, about 70 mg to about 800 mg, about 80 mg to about 800 mg, about 90 mg to about 800 mg, about 90 mg to about 750 mg, or about 90 mg to about 720 mg for each ingredient contained in the aforementioned pharmaceutical composition.

The aforementioned pharmaceutical composition can be for the reduction of fever due to upper respiratory tract infection. Upper respiratory tract infection (URTI) refers to a disease in which an acute infection occurs in the upper respiratory tract, that is, the nose, paranasal sinuses, pharynx and larynx, etc. The aforementioned upper respiratory tract infections can have symptoms such as nasal congestion, sore throat, tonsillitis, pharyngitis, laryngitis, paranasal sinusitis, otitis media, cold or any combination thereof. The aforementioned upper respiratory tract infection can be infection caused by virus, bacteria, fungus or parasite.

The aforementioned virus can be selected from the group composed influenza virus, rhinovirus, adenovirus, parainfluenza virus, respiratory syncytial virus, enterovirus and coronavirus. The aforementioned coronavirus can be severe acute respiratory syndrome (SARS) coronavirus, severe acute respiratory syndrome-coronavirus-2 (SARS-COV-2) or Middle East respiratory syndrome (MERS) coronavirus.

The aforementioned pharmaceutical composition can be for the prevention or treatment of coronavirus infection-19 (COVID-19) or influenza. The aforementioned pathogen of COVID-19 can be SARS-COV-2. Symptoms of the aforementioned COVID-19 can include fever (temperature of more than about 37.5° C.), cough, breathing difficulty, chills, muscle aches, headache, sore throat, loss of olfactory or gustatory senses, fatigue, loss of appetite, phlegm, digestive symptoms (nausea, vomiting and diarrhea, etc.), confusion, dizziness, runny or stuffy nose, hemoptysis, chest pain, conjunctivitis, skin symptoms or any combination thereof. The aforementioned influenza is a respiratory disease for which influenza virus invades the upper respiratory tract and causes viral infection. The aforementioned influenza can include fever of more than about 39ºC, cough, chest pain, runny or stuffy nose, sore throat, headache, body aches, myalgia, fatigue, weakness or any combination thereof.

The term “prevention” refers to any and all acts of inhibiting the manifestation of symptoms (example, fever and inflammation) or diseases (example, upper respiratory tract infection, COVID-19 and influenza), or delaying the onset thereof through the administration of the aforementioned pharmaceutical composition. The term “treatment” refers to any and all acts of improving the symptoms (example, fever and inflammation) or beneficially changing the course of a symptom or disease (example, upper respiratory tract infection, COVID-19, influenza) through the administration of the aforementioned pharmaceutical composition.

The aforementioned pharmaceutical composition can contain from about 30 mg to about 240 mg of artesunate or salts thereof, and from about 90 mg to about 720 mg of pyronaridine or salts thereof. The dose of the aforementioned artesunate or salts thereof can be from about 30 mg to about 220 mg, from about 30 mg to about 200 mg, from about 30 mg to about 180 mg, from about 30 mg to about 160 mg, from about 30 mg to about 140 mg, from about 30 mg to about 120 mg, from about 30 mg to about 100 mg, from about 30 mg to about 80 mg, from about 30 mg to about 60 mg, from about 40 mg to about 240 mg, from about 50 mg to about 240 mg, about 60 mg to about 240 mg, about 70 mg to about 240 mg, about 80 mg to about 240 mg, about 90 mg to about 240 mg, about 100 mg to about 240 mg, about 110 mg to about 240 mg, about 120 mg to about 240 mg, about 40 mg to about 200 mg, about 50 mg to about 160 mg, about 60 mg to about 140 mg, or about 60 mg to about 120 mg. The dose of the aforementioned pyronaridine or salts thereof can be about 90 mg to about 660 mg, about 90 mg to about 600 mg, about 90 mg to about 540 mg, about 90 mg to about 480 mg, about 90 mg to about 420 mg, about 90 mg to about 360 mg, about 110 mg to about 720 mg, 130 mg to about 720 mg, 150 mg to about 720 mg, 170 mg to about 720 mg, 180 mg to about 720 mg, 110 mg to about 660 mg, 130 mg to about 600 mg, 150 mg to about 540 mg, 160 mg to about 420 mg, or 160 mg to about 360 mg. The aforementioned pharmaceutical composition can be a compound preparation containing about 60 mg of artesunate or salts thereof and about 180 mg of pyronaridine or salts thereof.

The aforementioned pharmaceutical composition can be a preparation for single or individual administration. The aforementioned pharmaceutical composition can be a single or separate preparation. The aforementioned pharmaceutical composition can be a compound preparation containing about 60 mg of artesunate or salts thereof and about 180 mg of pyronaridine or salts thereof. The aforementioned pharmaceutical composition can be Pyramax® Tab.

The aforementioned pharmaceutical composition can be administered in a conventional formats including oral, transdermal, subcutaneous, rectal, intravenous, intra-arterial, intraperitoneal, intramuscular, intrasternal, topical or intradermal administrations. The dosage of the aforementioned pharmaceutical composition can be, for example, in the range of, about 0.001 mg/kg to about 100 mg/kg, about 0.01 mg/kg to about 10 mg/kg or about 0.1 mg/kg to about 1 mg/kg in an adult.

The aforementioned pharmaceutical composition can be administered once to twice a day, once to five times a week, once to five times for each 2-week interval or once to four times a month. 1, 2, 3 or 4 Pyramax tablets can be administered for each administration of the aforementioned pharmaceutical composition. The aforementioned pharmaceutical composition can be administered within 1 day, 2 days, 3 days, 4 days, 5 days, 6 days, 1 week, 2 weeks, 3 weeks, 1 month, 2 months or 3 months.

The aforementioned pharmaceutical composition can further contain publicly announced active ingredients having antipyresis activation function or pain suppressing activation function. For example, the aforementioned publicly announced active ingredients can be salicylic acid derivative (such as aspirin, etc.), aniline derivative (such as acetaminophen, phenacetin, etc.), virazorone derivative (such as sulpirin, aminopyrine, etc.), non-steroidal anti-inflammatory agent (such as indomethacin, mefenamic acid, ibuprofen, etc.) or a combination thereof.

The aforementioned preparation can including pharmaceutically allowable carrier. The aforementioned carrier is used to with meaning that includes excipients, diluents or adjuvants. The aforementioned carrier, for example, can those selected from the group compose of lactose, dextrose, sucrose, sorbitol, mannitol, xylitol, erythritol, maltitol, starch, acacia gum, alginate, gelatin, calcium phosphate, calcium silicate, cellulose, methyl cellulose, polyvinyl pyrrolidone, water, saline, buffers such as PBS, methyl hydroxybenzoate, propyl hydroxybenzoate, talc, magnesium stearate and mineral oil.

The aforementioned formulation can include filler, anticoagulant, lubricant, moisturizer, flavoring agent, emulsifier, preservative or a combination thereof.

The aforementioned formulation can be prepared in any dosage form in accordance with the conventional methods. For example, the aforementioned formulation can be formulated as an orally administered formulation (example, powder, tablet, capsule, syrup, pill or granule) or a parenteral formulation (example, injectable preparation). In addition, the aforementioned formulation can be manufactured as a systemic formulation or a topical formulation.

The aforementioned formulation can be an injection for subcutaneous, intramuscular or intravenous administration.

Another aspect provides a method of reducing fever including the steps of administering 30 mg to 240 mg of artesunate or salts thereof and 90 mg to 720 mg of pyronaridine or salts thereof to the subject once or twice a day.

Artesunate, pyronaridine, salts and administration are as described above.

The aforementioned individual can be an individual with fever or at risk of manifestation of fever. The aforementioned individual can be a mammal such as human, cow, horse, pig, dog, sheep, goat or cat.

Administration method can be oral or parenteral. The administration method, for example, can be through oral, transdermal, subcutaneous, rectal, intravenous, intra-arterial, intraperitoneal, intramuscular, intrasternal, topical, intranasal, intratracheal or intradermal routes. The aforementioned preparation can be administered systemically or locally, and can be administered singly or in combination with another pharmaceutically active compound.

Another aspect provides a method of preventing or treating COVID-19 that includes administration of 30 mg to 120 mg of artesunate or salts thereof and 90 mg to 360 mg of pyronaridine or salts thereof once or twice a day to the individual.

Individuals, artesunate, pyronaridine, salts, administration, coronavirus-infection, prevention and treatment are as set forth above.

### Effects of the Invention

According to the pharmaceutical composition containing artesunate or salts thereof and pyronaridine or salts thereof for antipyresis, antiinflammation or anti-viral action or for preventing or treating COVID-19, and method using the same, it is possible to achieve outstanding antipyretic effect with application of small dose with few adverse events and excellent treatment and prevention effect for febrile disease, upper respiratory tract infection, COVID-19 and influenza.

### BRIEF DESCRIPTION OF THE FIGURES

FIGS. 1 to 3 are graphs illustrating the number of patients with antipyresis according to the time of administration (t, h) of Pyramax® Tab. for febrile disease.

FIGS. 4 to 7 are graphs illustrating the number of patients with antipyresis according to the time of administration (t, h) of Pyramax® Tab. for fever and other upper respiratory tract infections.

### FORMAT FOR EXECUTION OF INVENTION

Hereinafter, this invention is described in more detail through embodiments. However, these embodiments are intended to illustrate the invention exemplarily and the scope of the invention is not limited to these embodiments.

### Embodiment 1. Confirmation of the Antipyretic or Fever Prevention Effect of Pyramax® Tab

(1) Confirmation of the Adverse Events in Accordance with Administration Dose of Pyramax® Tab.

It is known that adverse events such as nausea and vomiting can manifest when 4 tablets a day of Pyramax® Tab. is administered, which is the application method and dose for treatment of malaria.

To confirm the adverse events by differing the application methods and doses of Pyramax® Tab., adverse event in 420 people who took Pyramax® Tab. was checked as the subjects. Of these 420 subjects, 41 (9.8%) experienced adverse events while 90.2% had no adverse event at all. The majority of adverse events were nausea and vomiting. Specifically, 23 subjects (9.5%) experienced adverse events in the 1 tablet once a day group (n=240). In the 2 tablets once a day group (n=80), 3 subjects (3.75%) experienced adverse events, while the 3 tablets once a day group (n=86) had 8 subjects (9.3%) experiencing adverse events and the 4 tablet once a day group (n=14) had 7 subjects (50%).

To confirm the adverse effects of long-term administration of Pyramax® Tab., 108 people who took Pyramax® Tab. for up to 8 months were studied as the subjects for confirmation of adverse effects. Of these 108 subjects, there were no adverse event at all in the 2 tablets per week group (n=5), 1 tablet per week group (n=86) and 1 tablet per week group (n=16).

Therefore, 1, 2 and 3 administrations per day displayed lower level of adverse events in comparison to the application method of 4 administrations per day, and the probability of manifestation of adverse events at the time of administration of 4 tables once was substantially higher than that of the administration of lower doses. It was confirmed that there is lower level of adverse events even in the case of regular long-term administration.

Therefore, the adverse events of Pyramax can be reduced by administering the dose that is lower than 4 tablets once a day.

### (2) Antipyresis Effect on Febrile Diseases

### 1) Screening of the Patient Population

Patients (n=63) with body weight of more than 45 kg with the same symptoms of ‘fever’ were recruited. Patients were considered to have fever symptoms if they corresponded to more than 1 of the following criteria: Anorectal or tympanic temperature of more than 38.0° C., oral temperature of more than 37.5° C. and armpit temperature of more than 37.2° C.

Patients with fever were randomized into the 3 groups, namely, Group 1, Group 2 and Group 3 for which 1, 2 and 3 Pyramax® Tab. were administered once a day, respectively. In each group, The time taken for disappearance of fever (t, hours) from the time of administration of Pyramax® Tab. (time 0) was measured in each group.

TABLE 1

| Categories | Frequency (persons) | Percentage (%) |
|---|---|---|
| Group 1 | 22 | 34.9 |
| Group 2 | 21 | 33.3 |
| Group 3 | 20 | 31.7 |
| Total | 63 | 100.0 |

As illustrated in Table 1, 22 (34.9%) of the total of 63 subjects belonged to Group 1, followed by 21 (33.3%) in Group 2 and 20 (31.7%) in Group 3.

2) Time Taken for Antipyresis for Each Group of Pyramax® Tab. Administration Dose.

Frequency (persons) and percentage (%) according to the time taken for antipyresis in the total of 63 subjects administered with Pyramax® Tab. were computed and the results thereof are illustrated in Table 2.

TABLE 2

| Antipyresis | Frequency | |
|---|---|---|
| time (hours) | (persons) | Percentage (%) |
| 0 < t ≤ 12 | 4 | 6.3 |
| 12 < t ≤ 24 | 12 | 19.1 |
| 24 < t ≤ 48 | 32 | 50.8 |
| 48 < t ≤ 72 | 9 | 14.3 |
| 72 < t | 6 | 9.5 |
| Total | 63 | 100.0 |

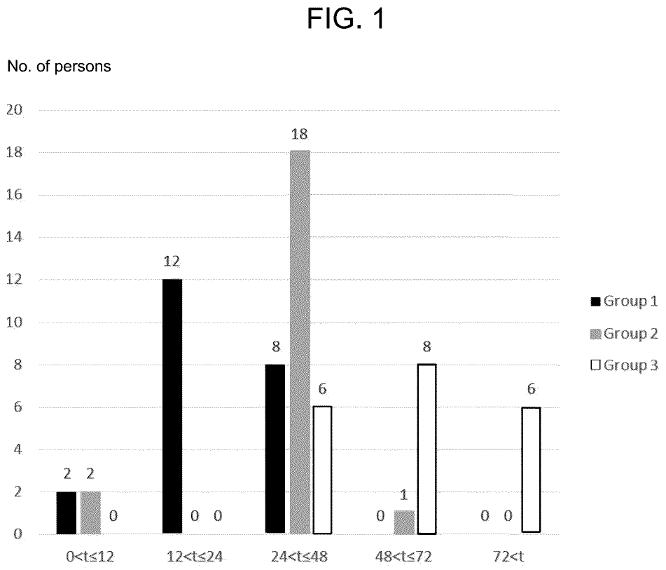

As illustrated in Table 2, 32 (50.8%) of the total of 63 subjects displayed antipyretic effect within 24 to 48 hours. Meanwhile, 12 patients (19.1%) displayed antipyretic effect within 12 to 24 hours and 9 patients (14.3%) within 48 to 72 hours, 4 patients (6.3%) within 0 to 12 hours and 6 patients (9.2%) after 72 hours. Antipyretic time was analyzed for each group according to the administration dose of Pyramax® Tab. and the results are illustrated in Table 3 and FIG. 1.

TABLE 3

| | Effects | | | | | |
|---|---|---|---|---|---|---|
| | 0 < | 12 < | 24 < | 48 < | | |
| Group | t ≤ 12 | t ≤ 24 | t ≤ 48 | t ≤ 72 | 72 < t | Total |
| Group 1 | 2 | 12 | 8 | 0 | 0 | 22 |
| | 9.1% | 54.5% | 36.4% | 0.0% | 0.0% | 100.0% |
| Group 2 | 2 | 0 | 18 | 1 | 0 | 21 |
| | 9.5% | 0.0% | 85.7% | 4.8% | 0.0% | 100.0% |
| Group 3 | 0 | 0 | 6 | 8 | 6 | 20 |
| | 0.0% | 0.0% | 30.0% | 40.0% | 30.0% | 100.0% |
| Total | 4 | 12 | 32 | 9 | 6 | 63 |
| | 6.3% | 19.1% | 50.8% | 14.3% | 9.5% | 100.0% |

(Fisher's exact test=58.507, p=0.000) As illustrated in Table 3 and FIG. 1, the antipyretic effect of Pyramax® Tab. according to the administered dose displayed statistically significant results (p<0.05). Of the total 63 subjects, the highest number of patients (18) belonged to Group 2, which displayed antipyretic effect within 24 to 48 hours. It was followed by 12 patients in Group 1 who displayed antipyretic effect within 12 to 24 hours. There were 8 patients in both the Group 3 and 1 for which antipyretic effect was displayed within 48 to 72 hours and within 24 to 48 hours, respectively. There were 6 patients each that displayed antipyresis effect within 24 to 48 hours and after 72 hours, respectively, in the Group 3, and 2 patients each in Groups 1 and 2 who displayed antipyretic effect within 0 (that is, immediately after administration) to 12 hours. There also was 1 patient in Group 2 who displayed antipyretic effect within 48 to 72 hours and there was no other patient.

### 3) Cross-Over Analysis Between Groups

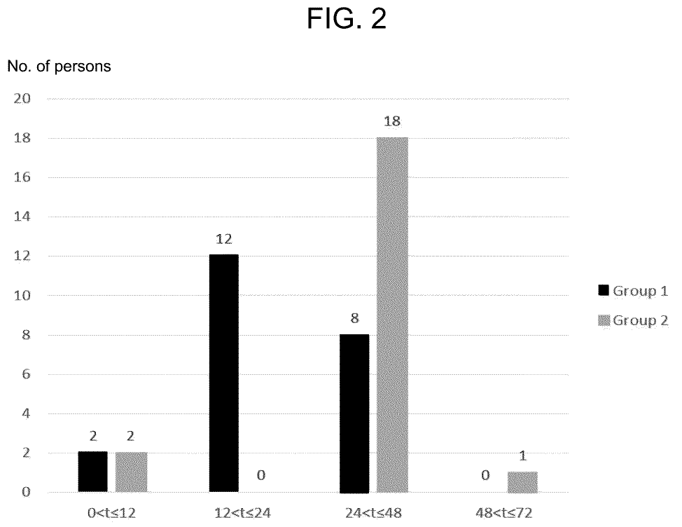

A crossover analysis was conducted between the Groups 1 and 2, and the results are illustrated in Table 4 and FIG. 2.

TABLE 4

| | Effects | | | | |
|---|---|---|---|---|---|
| | 0 < | 12 < | 24 < | 48 < | |
| Group | t ≤ 12 | t ≤ 24 | t ≤ 48 | t ≤ 72 | Total |
| Group 1 | 2 | 12 | 8 | 0 | 22 |
| | 9.1% | 54.5% | 36.4% | 0.0% | 100.0% |
| Group 2 | 2 | 0 | 18 | 1 | 21 |
| | 9.5% | 0.0% | 85.7% | 4.8% | 100.0% |
| Total | 4 | 12 | 26 | 1 | 43 |
| | 9.3% | 27.9% | 60.5% | 2.3% | 100.0% |

(Fisher's exact test=18.535, p=0.000) As illustrated in Table 4 and FIG. 2, the difference in the antipyretic effect between Group 1 and Group 2 was statistically significant (p<0.05). Of the total of 43 subjects, the highest number of patients belonged to Group 2, with 18 patients displaying antipyretic effect between 24 to 48 hours. There were 12 patients in Group 1 who displayed antipyretic effects within 12 to 24 hours, and 8 patients in Group 1 who displayed antipyretic effect within 24 to 48 hours. Both groups have 2 patients displaying antipyretic effect within 0 to 12 hours, while 1 patient in Group 2 displayed antipyretic effect within 48 to 72 hours.

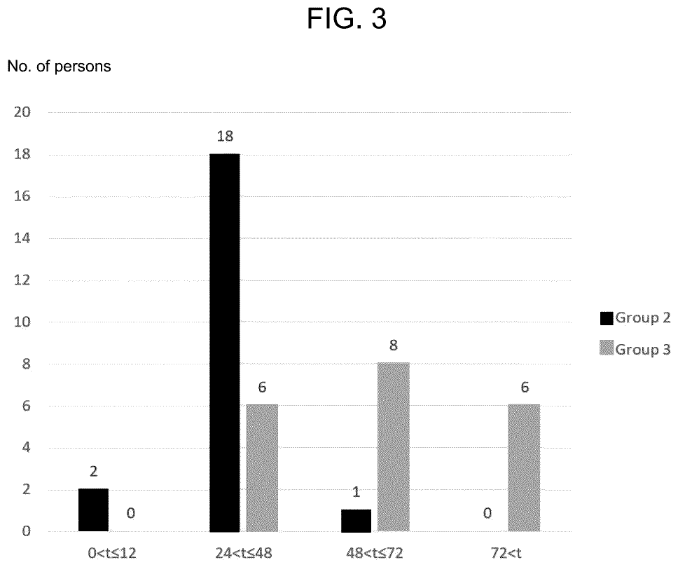

The results of the crossover analysis between Group 2 and Group 3 are illustrated in Table 5 and FIG. 3.

TABLE 5

| | Effects | | | | |
|---|---|---|---|---|---|
| | 0 < | 24 < | 48 < | | |
| Group | t ≤ 12 | t ≤48 | t ≤ 72 | 72 < t | Total |
| Group 2 | 2 | 18 | 1 | 0 | 21 |
| | 9.5% | 85.7% | 4.8% | 0.0% | 100.0% |
| Group 3 | 0 | 6 | 8 | 6 | 20 |
| | 0.0% | 30.0% | 40.0% | 30.0% | 100.0% |
| Total | 2 | 24 | 9 | 6 | 41 |
| | 4.9% | 58.5% | 22.0% | 14.6% | 100.0% |

(Fisher's exact test=19.123, p=0.000) As illustrated in Table 5 and FIG. 3, the difference in the antipyretic effect between Group 2 and Group 3 was statistically significant (p<0.05). Of the total of 41 subjects, the highest number of patients belonged to Group 2, with 18 patients displaying antipyretic effect within 24 to 48 hours. There were 8 patients in Group 3 who displayed antipyretic effect within 48 to 72 hours. Group 3 included 6 patients each who displayed antipyretic effect within 24 to 48 hours and after 72 hours, respectively. Group 2 had two patients who displayed antipyretic effect within 0 to 12 hours and 1 patient displaying antipyretic effect within 48 to 72 hours.

Therefore, subjects in Group 1 were able to relieve their fever faster than those in Group 2, and subjects in Group 2 faster than those in Group 3. The differences between these groups were statistically significant.

### (3) Antipyresis Effect on Fever and Other Upper Respiratory Tract Infections

Patients (n=22) with body weight of more than 45 kg with ‘fever and other symptoms of upper respiratory tract infection’ were recruited. After having categorized the patients into treatment groups according to application method and dose as described in Embodiment 1.(1.1.1), the time (t, hours) for fever to disappear was measured.

TABLE 6

| Categories | Frequency | Percentage |
|---|---|---|
| Group 1 | 10 | 45.5 |
| Group 2 | 7 | 31.8 |
| Group 3 | 5 | 22.7 |
| Total | 22 | 100.0 |

Out of a total of 22 subjects, Group 1 had the most patients with 10 (45.5%). It was followed by Group 2 with 7 patients (31.8%) and Group had the lowest number of patients at 5 (22.7%). Frequency (persons) and percentage (%) according to antipyresis time for the entire subjects who were administered with Pyramax® Tab. were calculated and the results are illustrated in Table 7.

TABLE 7

| Antipyresis | | |
|---|---|---|
| time (hours) | Frequency (persons) | Percentage (%) |
| 12 < t ≤ 24 | 4 | 18.2 |
| 24 < t ≤ 48 | 13 | 59.1 |
| 48 < t ≤ 72 | 2 | 9.1 |
| 72 < t | 3 | 13.6 |
| Total | 22 | 100.0 |

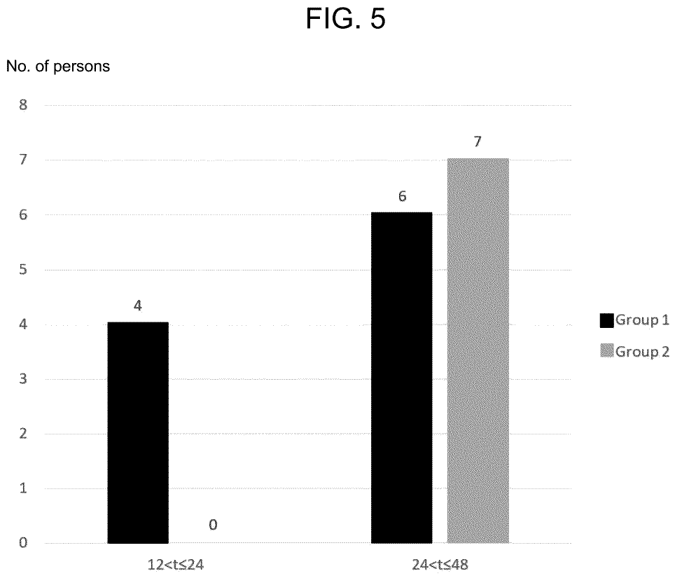

As illustrated in Table 7, the largest number of the subjects at 13 of the total of 22 patients (59.1%) displayed antipyretic effect within 24 to 48 hours. There were 4 patients (18.2%) displaying antipyretic effect within 12 to 24 hours, followed by 3 patients (13.6%) displaying antipyretic effect after 72 hours while the smallest No. of patients at 2 (9.1%) displayed antipyretic effect within 48 to 72 hours. The antipyretic time was analyzed for each group according to the administration dose of Pyramax® Tab. and the results are illustrated in Tables 8 to 10 and FIGS. 5 to 7.

TABLE 8

| | Effects | | | | |
|---|---|---|---|---|---|
| | 12 < | 24 < | 48 < | | |
| Group | t ≤ 24 | t ≤ 48 | t ≤ 72 | 72 < t | Total |
| Group 1 | 4 | 6 | 0 | 0 | 10 |
| | 40.0% | 60.0% | 0.0% | 0.0% | 100.0% |
| Group 2 | 0 | 7 | 0 | 0 | 7 |
| | 0.0% | 100.0% | 0.0% | 0.0% | 100.0% |
| Group 3 | 0 | 0 | 2 | 3 | 5 |
| | 0.0% | 0.0% | 40.0% | 60.0% | 100.0% |
| Total | 4 | 13 | 2 | 3 | 22 |
| | 18.2% | 59.1% | 9.1% | 13.6% | 100.0% |

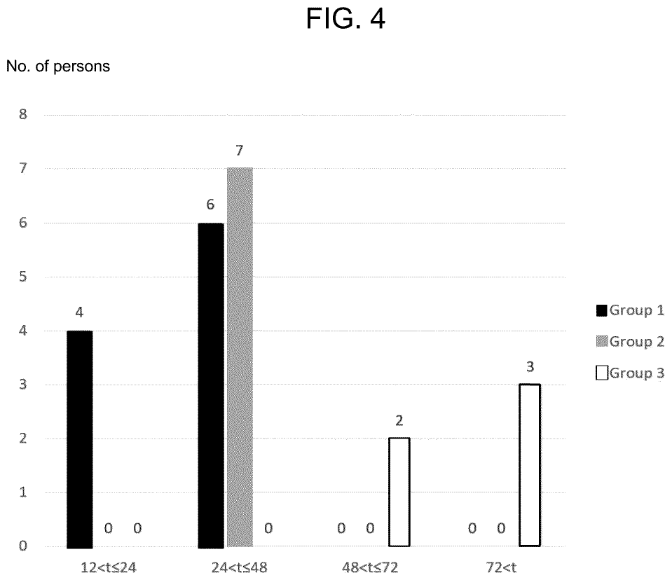

(Fisher's exact test=19.127, p=0.000) As illustrated in Table 8 and FIG. 4, the antipyretic effect of Pyramax® Tab. according to the administered dose was statistically significant (p<0.05). Of the total of 22 subjects, the highest number of subjects (7) belonged to Group 2, which displayed antipyretic effect within 24 to 48 hours. There were 6 patients in Group 1 who displayed antipyretic effect within the same time, followed by 4 patients in Group 1 who displayed antipyretic effect within 12 to 24 hours. Group 3 had 3 and 2 patients who displayed antipyretic effect for after 72 hours and within 48 to 72 hours, respectively.

TABLE 9

| | Effects | | |
|---|---|---|---|
| Group | 12 < t ≤ 24 | 24 < t ≤ 48 | Total |
| Group 1 | 4 | 6 | 10 |
| | 40.0% | 60.0% | 100.0% |
| Group 2 | 0 | 7 | 7 |
| | 0.0% | 100.0% | 100.0% |
| Total | 4 | 13 | 17 |
| | 23.5% | 76.5% | 100.0% |

(Fisher's exact test=3.662, p=0.103) As illustrated in Table 9 and FIG. 5, there was no statistically significant difference in the antipyretic effect of Pyramax® Tab. according to the administered dose between the Groups 1 and 2. Of the total of 17 subjects, Group 2 had the highest number of patients at 7 who displayed antipyretic effect within 24 to 48 hours. This was followed by 6 patients in Group 1 displaying an antipyretic effect within 24 to 48 hours and 4 patients in Group 1 within 12 to 24 hours.

TABLE 10

| | Effects | | | |
|---|---|---|---|---|
| Group | 24 < t ≤ 48 | 48 < t ≤ 72 | 72 < t | Total |
| Group 2 | 7 | 0 | 0 | 7 |
| | 100.0% | 0.0% | 0.0% | 100.0% |
| Group 3 | 0 | 2 | 3 | 5 |
| | 0.0% | 40.0% | 60.0% | 100.0% |
| Total | 7 | 2 | 3 | 12 |
| | 58.3% | 16.7% | 25.0% | 100.0% |

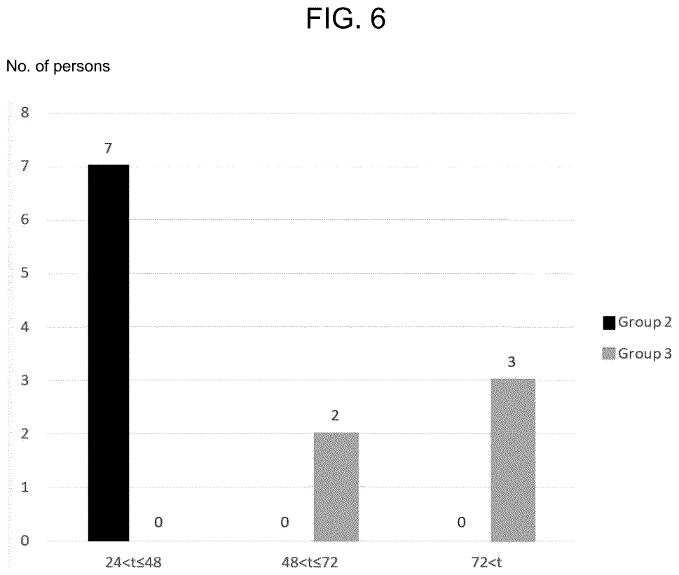

(Fisher's exact test=11.250, p=0.001) As illustrated in Table 10 and FIG. 6, the antipyretic effect of Pyramax® Tab. according to the dose administered was statistically significant (p<0.05). Of the total of 12 subjects, the number of patients in Group 2 who displayed antipyretic effect within 24 to 48 hours was the highest at 7. It was followed by 3 patients in Group 3 who displayed antipyretic effect after 72 hours, and 2 patients in Group 3 who displayed antipyretic effect within 48 to 72 hours.

TABLE 11

| | Effects | | | | |
|---|---|---|---|---|---|
| | 12 < | 24 < | 48 < | | |
| Group | t ≤ 24 | t ≤ 48 | t ≤ 72 | 72 < t | Total |
| Group 1 | 4 | 6 | 0 | 0 | 10 |
| | 40.0% | 60.0% | 0.0% | 0.0% | 100.0% |
| Group 3 | 0 | 0 | 2 | 3 | 5 |
| | 0.0% | 0.0% | 40.0% | 60.0% | 100.0% |
| Total | 4 | 6 | 2 | 3 | 15 |
| | 26.7% | 40.0% | 13.3% | 20.0% | 100.0% |

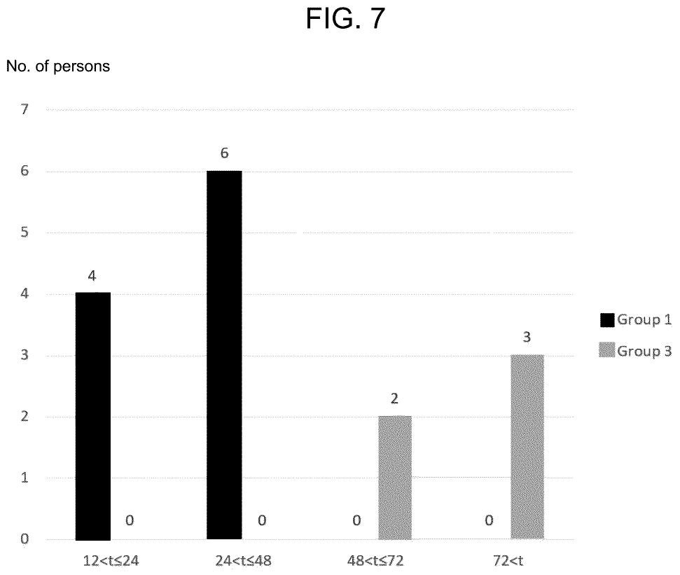

(Fisher's exact test=12.752, p=0.000) As illustrated in Table 11 and FIG. 7, the antipyretic effect of Pyramax® Tab. according to the administered dose was statistically significant (p<0.05). Of the total of 15 subjects, the number of patients in Group 1 who displayed antipyretic effect within 24 to 48 hours was the highest at 6. It was followed by Group 1 with 4 patients who displayed antipyretic effect within 12 to 24 hours.

Therefore, there was no statistically significant difference in the pace of disappearance of fever between Group 1 and Group 2. Both groups displayed faster disappearance of fever than that of Group 3, which was statistically significant.

(4) Application Method and Dose of Pyramax® Tab. For the Elimination of Fever

If it is to be as expected by making reference to the results of previous cellular experiments, and the application method/dose of malaria treatment medicine, fever should be eliminated most quickly in the group receiving three tablets at a time.

However, contrary to expectations, a dose lower than the dose used to treat malaria resulted in faster disappearance of fever symptoms. As such, it was confirmed that the appropriate dose of Pyramax® Tab. for antipyresis is 1 or 2 tablets once a day with the optimal dose being 1 tablet once a day.

Accordingly, it was confirmed that one or two tablets once a day provided outstanding antipyretic efficacy when administering Pyramax® Tab. to patients with fever and/or other symptoms of upper respiratory tract infection.

### (5) Preventive Effects on Feverish Colds

A family member (78 kg) had symptoms including high fever, chills, cough and diarrhea, and was administered with 3 Pyramax® Tabs once a day for 3 days. Since the improvement thereafter was not sufficient, the patient was administered with 4 tablets once a day for 2 more days.

3 Pyramax® Tabs were also administered once a day for 3 days to another member of the family (47 kg) without the symptom of feverish cold at the same time in addition to the aforementioned family member with feverish cold symptoms. However, the feverish cold of the family member with the aforementioned symptoms was not transmitted to other normal persons.

Therefore, it was confirmed that Pyramax has preventive effect in preventing feverish cold even if administered at a lower dose than that for malaria.

### Embodiment 2. Confirmation of the Preventive and Therapeutic Effects of Pyramax® Tab. On (COVID-19)

### (1) Therapeutic Efficacy on COVID-19

13 patients with confirmed COVID-19 were administered with Pyramax® Tab. In accordance with the dosage and application method stipulated in Table 12. Thereafter, the disappearance of COVID-19 symptoms or negative finding of SARS-COV-2 virus in RT-PCR test was confirmed.

TABLE 12

| | | Administration | |
|---|---|---|---|
| | | dose and | |
| No. | Symptoms | application method | Efficacy |
| 1 | Fever and | Administered 4 tablets | Fever disappeared after 5 days. |
| | chill | once a day for 4 days | Discharged after 10 days without |
| | | | confirmation of negative results |
| | | | in PCR test. |
| | | | It was diagnosed that there is no |
| | | | need for the test since the symptoms |
| | | | improved. |
| 2 | Body ache | Administered 3 tablets | Fever disappeared after 3 days |
| | and fever | once a day for 3 days | and was discharged after 5 days. |
| 3 | Fever | Administered 3 tablets | Fever disappeared after 3 days |
| | | once a day for 3 days | and was discharged after 7 days. |
| 4 | Fever and | Administered 3 tablets | Fever disappeared after 4 days and |
| | breathing | once a day for 3 days | was determined to be negative in |
| | difficulty | | SARS-CoV-2 PCR test after 1 week. |
| 5 | Fever | Administered 3 tablets | Fever disappeared after 1 day and |
| | | once a day for 3 days | was determined to be negative in |
| | | | SARS-CoV-2 PCR test after 1 week. |
| 6 | Asymptomatic | Administered 3 tablets | Was determined to be negative in |
| | | once a day for 3 days | SARS-CoV-2 PCR test after 1 week |
| 7 | Asymptomatic | Administered 3 tablets | Was determined to be negative in |
| | | once a day for 3 days | SARS-CoV-2 PCR test after 1 week |
| 8 | Asymptomatic | Administered 3 tablets | Was determined to be negative in |
| | | once a day for 3 days | SARS-CoV-2 PCR test after 1 week |
| 9 | Fever | Administered 1 tablet | Fever disappeared 3 days after |
| | and mild | once a day per week | diagnosis without further adminis- |
| | symptoms | for 9 weeks and was | tration and was determined to be |
| | | determined to be | negative in SARS-CoV-2 PCR test |
| | | infected during | after 10 days. (Others who was not |
| | | administration | administered with Pyramax ® Tab. |
| | | | took 20 days to be recovered.) |
| 10 | Fever | Administered 1 tablet | Improved after 3 weeks |
| | | once a day per week | |
| | | for 3 weeks (began | |
| | | administration after | |
| | | the infection) | |
| 11 | Fever | Administered 3 tablets | Fever disappeared 4 days was |
| | | once a day for 3 days | determined to be negative in SARS- |
| | | | CoV-2 PCR test after 4 days. |
| 12 | Fever and | Administered 3 tablets | Fever disappeared 3 days was |
| | coughing | once a day for 3 days | determined to be negative in SARS- |
| | | | CoV-2 PCR test after 3 days. |
| 13 | Asymptomatic | Administered 3 tablets | Was determined to be negative in |
| | | once a day for 3 days | SARS-CoV-2 PCR test after 6 days. |

As illustrated in Table 12, it was confirmed that administration of Pyramax® Tab. is therapeutically effective against COVID-19. In addition to the administration of 3 tablets once a day for 3 days, which is same as the conventional treatment method/dose for malaria, treatment was also found to be effective at a higher dose (4 tablets once a day for 4 days) and at a lower dose (1 tablet once a day for 1 week). More specifically, in 9 case, patients administered with the dose of 1 tablet per week for 9 weeks prior to the infection, which corresponds to less than 1/9 of the dose applied for the treatment of malaria, recovered in early stage without additional administration of Pyramax even after having been confirmed to be COVID-19 positive. In addition, in 10 cases, the patients recovered without being aggravated to severer conditions even when the administration began after having been infected at the dose of 1 tablet per week, which corresponds to less than 1/9 of the dose applied for the treatment of malaria.

Therefore, it was confirmed that it is possible to treat COVID-19 at doses significantly lower than the dose necessary to treat malaria.

### (2) Preventive Effect on COVID-19

Pyramax® Tab. was administered to normal persons who came in close contact with a confirmed COVID-19 case at the doses and application methods stipulated in Table 13. Thereafter, the occurrence of COVID-19 symptoms or the detection of SARS-COV-2 virus through RT-PCR test was checked.

TABLE 13

| | Administration | | |
|---|---|---|---|
| | dose and | | |
| No. | application method | Efficacy | Special remarks |
| 1 | Administered 4 tablets | Determined to be | Other passenger: Although |
| | once a day for 3 days. | negative in SARS- | mask was worn, Pyramax ® |
| | Came in close contact | CoV-2 PCR test | Tab. was not administered. |
| | by riding in the same | | Was determined to be |
| | vehicle with a person | | positive in SARS-CoV-2 |
| | with confirmed COVID- | | PCR test. |
| | 19 after 10 days | | |
| 2 | Was determined to be | Fever disappeared | Another family member |
| | COVID-19 positive | after 3 days without | who was administered |
| | while being admini- | additional administra- | with Pyramax ® Tab. |
| | stered with 1 tablet | tion of Pyramax ® | once a week was found to |
| | once a day per week | Tab. after having been | be COVID-19 negative. |
| | for 9 weeks. | diagnosed COVID-19 | |
| | | positive. | |
| | | Determined to be | |
| | | negative in SARS-CoV- | |
| | | 2 PCR test after 10 days. | |
| 3 | As a family member of | Determined to be | |
| | a person confirmed with | negative in SARS-CoV- | |
| | COVID-19, close | 2 PCR test. | |
| | contact occurred while | | |
| | being administered with | | |
| | 1 tablet once a day | | |
| | per week for 12 weeks. | | |

If Pyramax® Tab. is administered to normal person, SARS-COV-2 was detected even though the person came in close contact with confirmed COVID-19 case, and symptoms of COVID-19 infection did not aggravate to severer conditions even after the diagnosis as a confirmed COVID-19 case. Therefore, there is preventive effect on COVID-19 even with the administration of 1 Pyramax® Tab. once a day per week. Even if a person became infected while being administered at this dose, the symptoms of COVID-19 did not aggravate to severer conditions. In addition, there still was preventive effect against COVID-19 even after more than 10 days of the administration of Pyramax® Tab.

Accordingly, it was confirmed that Pyramax® Tab. at this low dose is effective in the prevention and treatment of COVID-19.

### Embodiment 3. Confirmation of the Effectiveness of Pyramax® Tab. In Treating Influenza

A patient diagnosed with influenza was administered with 3 Pyramax® Tabs once a day for 3 days, starting on the day of diagnosis. Influenza symptoms disappeared and the patient fully recovered after 6 days.

Therefore, it was confirmed that Pyramax® Tab. has a therapeutic effect on influenza as well.

What is claimed is:

1. A pharmaceutical composition containing artesunate or a salt thereof and pyronaridine or a salt thereof for antipyretic, anti-inflammatory or anti-viral action.

2. A pharmaceutical composition according to claim 1, wherein the pharmaceutical composition reduces fever caused by upper respiratory tract infection.

3. A pharmaceutical composition according to claim 2, wherein the pharmaceutical composition is for the aforementioned upper respiratory tract infection with symptoms including nasal congestion, sore throat, tonsillitis, pharyngitis, laryngitis, paranasal sinusitis, otitis media, cold or a combination thereof.

4. A pharmaceutical composition according to claim 2, where in the pharmaceutical composition is for the aforementioned upper respiratory tract infection caused by a virus selected from the group composed of influenza virus, rhinovirus, adenovirus, parainfluenza virus, respiratory syncytial virus, enterovirus and coronavirus.

5. A pharmaceutical composition according to claim 1, wherein the pharmaceutical composition is for the prevention or treatment of upper respiratory tract infection, COVID-19 or influenza.

6. A pharmaceutical composition according to claim 4, wherein the pharmaceutical composition is for corona viruses including severe acute respiratory syndrome-coronavirus-2 (SARS-COV-2).

7. A pharmaceutical composition according to claim 1, wherein the pharmaceutical composition contains salt of pyronaridine, which is pyronaridine phosphate.

8. A pharmaceutical composition according to claim 1, wherein the pharmaceutical composition contains 30 mg to 240 mg of artesunate or salts thereof and 90 mg to 720 mg of pyronaridine or salts thereof.

9. A pharmaceutical composition according to claim 1, wherein the pharmaceutical composition is administered once or twice a day.

10. A pharmaceutical composition according to claim 1, wherein the pharmaceutical composition contains 30 mg to 240 mg of artesunate or salts thereof and 90 mg to 720 mg of pyronaridine or salts thereof, and is administered once or twice a day.

11. A method of reducing fever that includes the stage of administering pharmaceutical composition containing 30 mg to 240 mg of artesunate or salts thereof and 90 mg to 720 mg of pyronaridine or salts thereof once or twice a day to an individual.

12. A method of preventing or treating an upper respiratory tract infection, COVID-19 or influenza that includes the stage of administering pharmaceutical composition containing 30 mg to 120 mg of artesunate or salts thereof and 90 mg to 360 mg of pyronaridine or salts thereof once or twice a day to an individual.
